(19) United States
(12) Patent Application Publication
(10) Pub. No.: US 2007/0276037 A1
(43) Pub. Date: Nov. 29, 2007

(54) Cytoprotective Effects of Ethyl Pyruvate

(76) Inventor: Yi-Ping Joseph Woo, Lafayette Hills, PA (US)

Correspondence Address: PEARL COHEN ZEDEK LATZER, LLP, 1500 BROADWAY 12TH FLOOR, NEW YORK, NY, 10036, US

(21) Appl. No.: 10/577,256

(22) PCT Filed: Oct. 27, 2004
(86) PCT No.: PCT/US04/35445

§ 371 Date: May 16, 2007

Related U.S. Application Data
(60) Provisional application No. 60/514,781, filed on Oct. 27, 2003.
(60) Provisional application No. 60/555,839, filed on Mar. 24, 2004.

(51) Int. Cl. A61K 31/22; A61P 9/10; C12N 5/00

(52) U.S. Cl. 514/546; 435/375

(57) ABSTRACT

The present invention provides methods for conferring cytoprotection and treating, suppressing or reducing the incidence of ischemia. The methods make use of an ester or amide of an alpha-ketoalkanoic acid, such as ethyl pyruvate, or compositions comprising the same.

### FIELD OF THE INVENTION

This invention provides for the use of ester or amide of an alpha-ketoalkanoic acid, such as ethyl pyruvate in cytoprotection and prevention or inhibition of ischemia. Methods for treating, preventing or reducing stroke-related injury, myocardial infarction or damage caused thereby are disclosed.

### BACKGROUND OF THE INVENTION

Myocardial dysfunction and injury following ischeria are attributed to multiple factors, of which metabolic depletion of high-energy phosphates and formation of reactive oxygen species (ROS) are perhaps most predominant. During ischemia, without citric acid cycle contribution, energy requirements are poorly met by anaerobic glycolysis.

Another major mechanism of injury involves the generation of ROS during reperfusion via multiple reactions, resulting in the production of the following reactive species: superoxide anion, hydrogen peroxide, peroxynitrite and hydroxyl radical. These reactive species produce a spectrum of cellular injury via multiple mechanisms including membrane destabilization, mitochondrial disruption, and metabolic derangement. Native antioxidant enzyme systems exist, which convert ROS, to water and oxygen, including superoxide dismutase, catalase and glutathione peroxidase, however these systems are not always sufficient. There is a current need for strategies to augment these systems and provide greater protection.

Off-pump coronary artery bypass grafting is associated with transient periods of myocardial ischemia during revascularization. Such ischemia puts the myocardium at risk for contractile dysfunction and injury associated in part by the metabolic depletion of high-energy phosphates. In addition, reperfusion following revascularization is associated with myocardial oxidative injury mediated by reactive oxygen species. Current strategies have not provided optimal protection from the consequences of this life-saving procedure.

Pyruvate, in the presence of hydrogen peroxide, decarboxylates, yielding acetate, water, and carbon dioxide. Pyruvate is capable of scavenging hydroxyl radicals, and directly increases sarcoplasmic reticular ATPase activity and hence calcium cycling efficiency. Unfortunately, the therapeutic potential of exogenously administered pyruvate is significantly limited by extreme aqueous instability.

### SUMMARY OF THE INVENTION

This invention provides, in one embodiment, a method for conferring cytoprotection, comprising contacting a cell with an amount of ethyl pyruvate, effective to confer cytoprotection. In one embodiment, the cell is within or isolated from an organ which is ischemic, or in another embodiment, within or isolated from an organ, which is undergoing, or at risk of oxidative damage or in another embodiment, within or isolated from myocardium. In another embodiment, the cell is implanted within a subject following contact with said effective amount of ethyl pyruvate. In another embodiment, the cell is within a population of cells which will be transplanted to a subject.

In another embodiment, this invention provides a method for conferring cytoprotection, comprising contacting a cell with an amount of an ester or amide of an alpha-ketoalkanoic acid, effective to confer cytoprotection. In one embodiment, the method comprises contacting said cell with an ester of an alpha-ketoalkanoic acid. In another embodiment, the cell is within or isolated from an organ which is ischemic, or in another embodiment, within or isolated from an organ, which is undergoing, or at risk of oxidative damage or in another embodiment, within or isolated from myocardium. In another embodiment, the cell is implanted within a subject following contact with said effective amount of the ester or amide of an alpha-ketoalkanoic acid. In another embodiment, the cell is within a population of cells which will be transplanted to a subject.

In another embodiment, the ethyl pyruvate treats, inhibits or reduces the incidence of oxidative damage to the cell, or in another embodiment, augments cellular metabolism.

In another embodiment, the ester or amide of an alpha-ketoalkanoic acid inhibits or reduces the incidence of oxidative damage to the cell, or in another embodiment, augments cellular metabolism.

In another embodiment, this invention provides a method for treating, preventing or reducing ischemic damage to the heart, in a subject, comprising administering ethyl pyruvate to said subject in an amount effective to treat, reduce or prevent ischemic damage to the heart. In one embodiment, the ethyl pyruvate is administered to the subject prior to a surgical procedure having potential to cause cardiac ischemic damage. In another embodiment, the ethyl pyruvate is administered to the subject during a surgical procedure having potential to cause cardiac ischemic damage. In another embodiment, ethyl pyruvate is administered to the subject following a surgical procedure having potential to result in cardiac ischemic damage.

In another embodiment, this invention provides a method for treating, preventing or reducing ischemic damage to the heart, in a subject, comprising administering an ester or amide of an alpha-ketoalkanoic acid to said subject in an amount effective to treat, reduce or prevent ischemic damage to the heart. In another embodiment, the ester or amide of an alpha-ketoalkanoic acid is administered to the subject prior to a surgical procedure having potential to cause cardiac ischemic damage. In another embodiment, the ester or amide of an alpha-ketoalkanoic acid is administered to the subject during a surgical procedure having potential to cause cardiac ischemic damage. In another embodiment, the ester or amide of an alpha-ketoalkanoic acid is administered to the subject following a surgical procedure having potential to result in cardiac ischemic damage.

In one embodiment, the subject is in need of said treatment due to an angina condition which comprises chronic stable angina, unstable angina or post myocardial infarction angina. In another embodiment, the subject is in need of such treatment due to acute myocardial infarction

In another embodiment, the invention provides a method for conferring cytoprotection in a subject, comprising administering an effective amount of ethyl pyruvate to said subject, wherein said amount is sufficient to confer cytoprotection, In one embodiment, the subject is undergoing a surgical procedure, which in one embodiment is a vascular procedure. In one embodiment, the procedure is coronary artery bypass graft surgery (CABG). Furthering another embodiment, the procedure is off-pump coronary artery bypass graft surgery. In another embodiment, the procedure involves the use of a heart-lung bypass machine. In another embodiment, the procedure is for cerebral vascular or percutaneous coronary intervention. In another embodiment, the cytoprotection is conferred to cells at risk for, or having an injury or damage due to reperfusion. In another the procedure is conducted in the thoracic cavity, and in another embodiment, the ethyl pyruvate confers cytoprotection to cells of the spinal cord.

In another embodiment, the invention provides a method for conferring cytoprotection in a subject, comprising administering an effective amount of ester or amide of an alpha-ketoaloic acid to said subject, wherein said amount is sufficient to confer cytoprotection. In one embodiment, the subject is undergoing a surgical procedure, which in one embodiment is a vascular procedure. In one embodiment, the procedure is coronary artery bypass graft surgery (CABG). In another embodiment, the procedure is off-pump coronary artery bypass graft surgery. In another embodiment, the procedure involves the use of a heart-lung bypass machine. In another embodiment, the procedure is for cerebral vascular or percutaneous coronary intervention. In another embodiment, the cytoprotection is conferred to cells at risk for, or having an injury or damage due to reperfusion. In another the procedure is conducted in the thoracic cavity, and in another embodiment, the ester or amide of an alpha-ketoalkanoic acid confers cytoprotection to cells of the spinal cord.

In another embodiment, this invention provides a method for treating, suppressing or reducing the incidence of stroke-related injury, in a subject, comprising administering ethyl pyruvate to said subject in an amount effective to treat, prevent or reduce stroke-related injury in said subject.

In another embodiment, this invention provides a method for treating, suppressing or reducing the incidence of stroke-related injury, in a subject, comprising administering an ester or amide of an alpha-ketoalkanoic acid to said subject in an amount effective to treat, prevent or reduce stroke-related injury in said subject

In another embodiment, this invention provides a method of treating, suppressing or inhibiting the incidence of myocardial infarction or damage caused by myocardial infarction in a subject, the method comprising administering ethyl pyruvate to a subject at risk for or undergoing myocardial infarction, in an amount effective at treating, suppressing or inhibiting the incidence of myocardial infarction or damage caused by myocardial infarction.

In another embodiment, this invention provides a method of treating, suppressing or inhibiting the incidence of myocardial infarction or damage caused by myocardial infarction in a subject, the method comprising administering an ester or amide of an alpha-ketoalkanoic acid to a subject at risk for or undergoing myocardial infarction, in an amount effective at treating, suppressing or inhibiting the incidence of myocardial infarction or damage caused by myocardial infarction.

### BRIEF DESCRIPTION OF THE DRAWINGS

FIG. 1 demonstrates ATP levels measured bioluminescently in ischemic myocardium 10 minutes after the initiation of ischemia. Significantly higher levels of ATP were observed in ethyl pyruvate-treated hearts (n=5) compared with controls (n=5).

FIG. 2 demonstrates lipid peroxidation in myocardium following ischemia and reperfusion. Lipid peroxidation in remote nanischeric myocardium was equivalent between control (n=6) and ethyl pyruvate (n=6) hearts. Ischemia increased the level of liqid peroxidation in the control hearts. The administration of ethyl pyruvate decreased the level of lipid peroxidation in the ischemic myocardium when compared with control hearts.

FIG. 3 Lipid peroxide levels in both ischemic and non-ischemic myocardium (n=5, in both treatment groups). Non-ischemic myocardium was not significantly different between treatment groups. Lipid peroxides were significantly reduced in the ethyl pyruvate treated group as compared to controls (70.4±2.6 nmol/g vs. 81.8±2.4nmol/g, p=0.04).

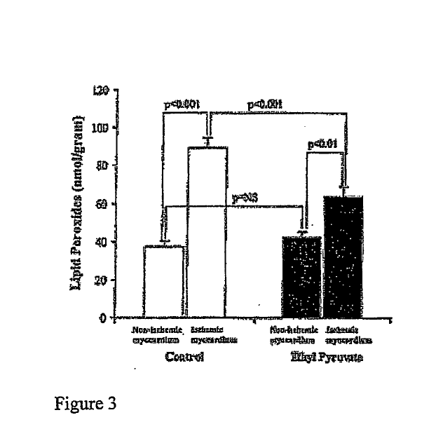

FIG. 4 demonstrates myocardial infarction percentage measured by infarction area IA/area at risk (AAR) following ischemia and reperfusion Significant differences in left ventricular infarction were observed between animals receiving ethyl pyruvate (n=15) and the control group (n=15).

FIG. 5 demonstrates maximum left ventricular (LV) pressure expressed as a percentage of baseline. Maximum LV pressure was significantly greater throughout ischemia and reperfusion in the ethyl pyruvate treatment group compared to controls. Asterisk (*) denotes statistical significance of p<0.05.

FIG. 6 demonstrates the dP/dtax, a measure of contractility, expressed as a percentage of baseline. dP/dtmax was significantly greater after 8 minutes of ischemia and throughout reperfusion in the ethyl pyruvate treatment group compared to controls. Asterisk (*) denotes statistical significance of p<0.05.

FIG. 7 demonstrates cardiac output expressed as a percentage of baseline. Cardiac output was significantly greater throughout ischemia and reperfusion in the ethyl pyruvate treatment group compared to controls. Asterisk (*) denotes statistical significance of p<0.05.

FIG. 8 provides a composite representation of end-systolic pressure-volume relationships (ESPVR) in native animals (n=15) and in control (n=15) and ethyl pyruvate-treated (n=15) animals following ischemia and reperfusion. For native animals, mean±SEM slope, x-axis intercept (x-int), and correlation coefficient (r) were: 1.27±0.12, 10.4±2.0, and 0.97±0.07, respectively. For control animals, the values were: slope=0.59±0.2, x-int=9.9±24.0, r=0.68±0.13; for ethyl pyruvate-treated animals, these values were: slope=1.09±0.22, x-int=4.1±19.9, r=0.89±0.03. Comparing the slopes of the control and ethyl pyruvate hearts demonstrates a statistically significant improvement in contractility with the administration of ethyl pyruvate (P=0.02). Composite ESPVR for the native animals (n=15) was 1.27 (ESV-10.39), control (n=15) was 0.59 (ESV-9.88), and ethyl pyruvate (n=15) was 1.08 (ESV-4.0B). Significant differences in the slope of the ESPVR were found between ethyl pyruvate and control animals.

### DETAILED DESCRIPTION OF THE INVENTION

This invention provides, in one embodiment, applications of the augmentation of glycolytic substrates mediated by ethyl pyruvate, the antioxidant properties of ethyl pyruvate, or a combination thereof.

This invention provides, in another embodiment, applications of the augmentation of glycolytic substrates mediated by an ester or amide of an alpha-ketoalkanoic acid, the antioxidant properties of an ester or amide of an alpha-ketoalkanoic acid, or a combination thereof.

Ethyl pyruvate augmentation of glycolytic substrates was exemplified herein, in Example 1, wherein tissue ATP levels assayed in the ischeric myocardial territory 10 minutes after LAD snaring, increasing ATP generation The period of time chosen enabled depletion of potentially confounding myocardial high-energy phosphate reserves, and ensured dependence upon anaerobic glycolysis, which mimics many clinically relevant conditions.

Diminution of myocardial oxidative injury in response to ethyl pyruvate was exemplified herein. Lipid peroxidation was reduced in treated tissue (Example 2), in an assay system, which provides a direct measure of free radical tissue injury.

In one embodiment, an ester of an alpha-ketoalkanoic acid comprises for example, a C₃-C₈ straight-chained or branched alpha-ketoalkanoic acid. Other examples include alpha-keto-butyrate, alpha-ketopentanoate, alpha-keto-3-methyl-butyrate, alpha-keto-4-methyl-pentanoate or alpha-keto-hexanoate. In one embodiment, the ester of an alpha-ketoallaoic acid comprises ethyl pyruvate.

In one embodiment, a variety of groups such as alkyl, aralkyl, alkoxyalkyl, carbalkoxyalkyl or acetoxyalkyl are suitable for the ester position of the molecule. In one embodiment, they comprise: ethyl, propyl, butyl, carbmethoxymethyl (—CH₂COOCH₃), carbethoxymethyl (—CH₂COOCH₂CH₃), acetoxymethyl (—CH₂0C(O)CH₃), carbmethoxyethyl (—CH₂CH₂COOCH₃), carbethoxyethyl (—CH₂CH2COOCH₂CH₃), methoxymethyl (—CH₂OCH₃) and ethoxymethyl (—CH₂OCH₂CH₃). In one embodiment, an ethyl esters is used. Thiolesters (e.g., wherein the thiol portion is cysteine or homocysteine) and glyceryl esters (e.g., wherein one or mote of the alcohol groups on glycerol are replaced with an alpha-ketoalkanoate group) represent additional embodiments.

In another embodiment, alpha-ketoalkanoic esters suitable for use in the disclosed methods comprise ethyl pyruvate, propyl pyruvate, carbmethoxymethyl pyruvate, acetoxymethyl pyruvate, carbethoxymethymethyl pyruvate, ethoxymethyl pyruvate, ethyl alpha-keto-butyrate, ethyl alpha-keto-pentanoate, ethyl alpha-keto-3-methyl-butyrate, ethyl alpha-keto-4-methyl-pentanoate, or ethyl alpha-keto-hexanoate. In one embodiment, ethyl pyruvate is may be the alpha-keto-acid ester used.

In another embodiment, an amide of alpha-ketoalkanoic acids for use in the methods of the present inventions include compounds having the following structural formula: RCOCONR₁R₂. R is an alkyl group; R1 and R₂ are independently —H, alkyl, aralkyl, alkoxyalkyl, carbalkoxyalkyl or —CHR₃COOH (i.e. an “amino acid amide” of an alpha-ketoalkanoic acid); and R3 is the side chain of a naturally occurring amino acid. In one embodiment, the amide of an alpha-ketoalkanoic acids is a pyruvamide.

In one embodiment, suitable alkyl groups include C₁-C₈ straight chained or branched alkyl group, preferably C₁-C₆ straight chained alkyl groups.

In another embodiment, suitable aryl groups include carbocyclic (e.g., phenyl and naphthyl) and heterocyclic (e.g., furanyl and thiophenyl) aromatic groups, which in another embodiment are phenyl groups.

In another embodiment, an aloxy group is —OR₄, wherein R4 is an alkyl group, as defined above. An alkoxyalkyl group is an alkyl group substituted with —OR4.

In another embodiment, an aralkyl group is —XY, wherein X is an alkyl group and Y is an aryl group, both as defined above.

In another embodiment, a carboxyalkyl group is an alkyl group substituted with —COOH. A carbalkoxyalkyl group is an alkyl group substituted with —-C(O)OR, wherein R is an alkyl group, as defined above.

In another embodiment, an acyl group is —C(O)—R, wherein R is an alkyl group, as defined above.

In another embodiment, an acetoxy alkyl group is an alkyl group substituted with —O—C(O)—R, wherein R is an alkyl group, as defined above.

In one embodiment, this invention provides a method for conferring cytoprotection, comprising contacting a cell with an amount of ethyl pyruvate, effective to confer cytoprotection.

In one embodiment, this invention provides a method for conferring cytoprotection, comprising contacting a cell with an amount of an ester or amide of an alpha-ketoalkanoic acid, effective to confer cytoprotection.

In one embodiment, the tern “cytoprotection” refers to protection of cells from damage and death in conditions where intrinsic or extrinsic damage has occurred. In another embodiment, the term “cytoprotection” refers to slowing, halting or preventing the injury, deterioration and/or death of a cell or a population of cells. Such injury, deterioration and/or death may be precipitated, in another embodiment, by one or more external factors or by intrinsic factors including apoptosis or by a combination of such factors. In one embodiment, cytoprotection refers to protection from oxidative injury.

In one embodiment, ethyl pyruvate confers cytoprotection by directly diminishing injury or, in another embodiment, indirectly diminishing injury. In another embodiment, indirect diminution of injury, or cytoprotection, is mediated via augmentation of cellular metabolism.

In another embodiment, the ester or amide of an alpha-ketoalkanoic acid confers cytoprotection by directly diminishing injury or, in another embodiment, indirectly diminishing injury.

In one embodiment contacting a cell with ethyl pyruvate confers cytoprotection. In one embodiment, the term “cell” is to be understood as comprising an isolated single cell, or cells in culture, within tissues or organs. In one embodiment the cell is a cardiocyte, or in another embodiment, a neuron, or in another embodiment a stem cell. In one embodiment, the cell is found within the heart, or in another embodiment, the brain, or in another embodiment, the spinal cord, or in another embodiment, the liver, or in another embodiment, the kidney, or in another embodiment, the pancreas, or in another embodiment, the bone marrow, or in another embodiment, a sensory organ.

In one embodiment contacting a cell with an ester or amide of an alpha-ketoalkanoic acid confers cytoprotection.

In one embodiment, the term “contacting a cell” refers to both direct and indirect exposure of the cell ethyl pyruvate a composition comprising the same. In one embodiment, contacting a cell may comprise direct injection of the cell through any means well known in the art such as microinjection. It is also envisaged, in another embodiment, that supply to the cell is indirect, such as via provision in a culture medium that surrounds the cell, for cells in culture. In another embodiment, the cell is within an organ, and contacting a cell may refer to the suspension of the organ with ethyl pyruvate, or a composition comprising the same, or in another embodiment, the organ is perfumed with ethyl pyruvate or a composition comprising the same.

In one embodiment, the term “contacting a cell” refers to both direct and indirect exposure of the cell to the ester or amide of an alpha-ketoalkanoic acid or a composition comprising the same. In one embodiment, contacting a cell may refer to the suspension of the organ with the ester of amide of an alpha-ketoalkanoic acid or a composition comprising the same, or in another embodiment, the organ is perfused with the ester or amide of an alpha-ketoalkanoic acid or a composition comprising the same.

In another embodiment, contacting the cell is conducted in vitro, or in another embodiment, contacting the cell is conducted in vivo. In one embodiment, in vivo contact may be direct, or in another embodiment, indirect In one embodiment, direct in vivo contact may comprise localized injection or catheterization. In another embodiment, indirect in vivo contact may comprise systemic administration, via any number of means well known in the art, such as, oral administration, intravenous injection, aerosol, intranasal administration, and other routes of administration, some embodiments of which are further described herein below.

In another embodiment, the invention provides a method for conferring cytoprotection in a subject, comprising administering an effective amount of ethyl pyruvate to said subject, wherein said amount is sufficient to confer cytoprotection In one embodiment, the subject is undergoing a surgical procedure, which in one embodiment is a vascular procedure. In one embodiment, the procedure involves the use of a heart-lung bypass machine. In another embodiment, the procedure is for cerebral vascular or percutaneous coronary intervention. In another embodiment, the cytoprotection is conferred to cells at risk for, or having an injury or damage due to reperfusion In another the procedure is conducted in the thoracic cavity, and in another embodiment, the ethyl pyruvate confers cytoprotection to cells of the spinal cord.

In another embodiment, the invention provides a method for conferring cytoprotection in a subject, comprising administering an effective amount of an ester or amide of an alpha-ketoalkanoic acid to said subject, wherein said amount is sufficient to confer cytoprotection. In one embodiment, the ester or amide of an alpha-ketoalkanoic acid confers cytoprotection to cells of the spinal cord.

In another embodiment, the procedure is coronary artery bypass graft surgery (CABG). CABG surgery can be utilized to treat a blockage or restriction in the blood flow leading to the heart and is commonly known as a “heart bypass” operation In a CABG surgery, a surgeon may remove a portion of a vein from another part of the body to use as a graft and installs the graft at points which bypass the obstruction to restore normal blood flow to the heart In one embodiment, the CABG surgery is off-pump coronary artery bypass graft surgery. Off-pump CABG is similar to conventional CABG surgery but the procedure is performed while the heart is still beating. In off-pump CABG, use of cardiopulmonary bypass machine and stopping of the heart are not required.

In another embodiment, cytoprotection is conferred by the methods of this invention to cells within or isolated from an organ which is undergoing, or at risk of oxidative damage. In another embodiment, the ethyl pyruvate inhibits or reduces the incidence of oxidative damage to the cell.

In another embodiment the ester or amide of an alpha-ketoalkanoic acid inhibits or reduces the incidence of oxidative damage to the cell.

Oxidative damage may arise form a failure to quench free radical reactions, as these reactions result in damage to macromolecules, lipid peroxidation, and generation of toxic compounds.

Administration of ethyl pyruvate may be accompanied by the administration of other cytoprotection compounds. Treatment may be administered in vivo that is to the patient or ex vivo to a sample of isolated tissue outside the body, which is destined for asplantation. The treated subject or tissue protected according to the invention is not species-restricted but may be applied to tissue from any animal, including any mammal, such as domestic animals, for example, pigs, cows and sheep, as well as primates including humans. In one embodiment, the cell is implanted within a subject following contact with said effective amount of ethyl pyruvate. In another embodiment, the cell is within a population of cells, which will be transplanted to a subject. In another embodiment, an entire organ or tissue is contacted with ethyl pyruvate, and implanted in a subject. According to this aspect of the invention, and in one embodiment, the cytoprotection effect of ethyl pyruvate is useful in preventing oxidative damage to the cells/tissue/organ for implantation, or in another embodiment.

In another embodiment, administration of the ester or amide of an alpha-ketoallaoic acid may be accompanied by the administration of other cytoprotection compounds. In one embodiment, the cell is implanted within a subject following contact with said effective amount of the ester or amide of an alpha-ketoalkanoic acid. In another embodiment, the cell is within a population of cells, which will be transplanted to a subject. In another embodiment, an entire organ or tissue is contacted with the ester or amide of an alpha-ketoalkanoic acid, and implanted in a subject According to this aspect of the invention, and in one embodiment, the cytoprotection effect of the ester or amide of an alpha-ketoalkanoic acid is useful in preventing oxidative damage to the cells/tissue/organ for implantation, or in another embodiment.

In one embodiment, ethyl pyruvate, or a composition comprising the same is administered to a subject, via the oral, intramuscular, transdermal, nasal, buccal, intravenous, rectal, intramuscular or subcutaneous route, or at any site where cytoprotection is desired. Methods of administering ethyl pyruvate, or a composition comprising the same may be by unit dose or by controlled release vehicles.

In one embodiment, the ester or amide of an alpha-ketoalkanoic acid, or a composition comprising the same is administered to a subject, via the oral, intramuscular, transdermal, nasal, buccal, intravenous, rectal, intramuscular or subcutaneous route, or at any site where cytoprotection is desired. Methods of administering the ester or amide of an alpha-ketoalkanoic acid, or a composition comprising the same may be by unit dose or by controlled release vehicles.

The use of ethyl pyruvate, or a composition comprising the same, results in one embodiment of this invention, in an improved outcome for cell, organ, and tissue transplantation by reducing the damage to the tissue that leads to decreased viability of the cells.

The use of the ester or amide of an alpha-ketoalkanoic acid, or a composition comprising the same, results in one embodiment of this invention, in an improved outcome for cell, organ, and tissue transplantation by reducing the damage to the tissue that leads to decreased viability of the cells.

Tissues that are protected by the methods of the invention may be derived from children, adult or fetuses and include, but are not limited to, stem cells, blood and all of its components, including erythrocytes, leukocytes, platelets and serum, central nervous tissue, including brain and spinal cord tissue, neurons, and glia; peripheral nervous tissue, including ganglia, posterior pituitary gland, adrenal medulla, and pineal; connective tissue, skin, ligaments, tendons, and fibroblasts; muscle tissue, including skeletal, smooth and cardiac tissues or the cells therefrom; endocrine tissue, including anterior pituitary gland, thyroid gland, parathyroid gland, adrenal cortex, pancreas and its subparts, testes, ovaries, placenta, and the endocrine cells that are a part of each of these tissues; blood vessels, including arteries, veins, capillaries and the cells from these vessels: lung tissue; heart tissue and whole organ; heart valves; liver; kidney; intestines; bone, including osteocytes, osteoblasts and osteoclasts; immune tissue, including blood cells, bone marrow and spleen; eyes and their parts; reproductive tract tissues; or urinary tract tissue.

In an embodiment of the invention, the methods of this invention utilize an effective dose of ethyl pyruvate, wherein low concentrations of ethyl pyruvate in culture fluid or in body fluids may result. In one embodiment, the methods of this invention employ a dosage of ethyl pyruvate such that plasma concentrations of 200 nM or less and greater than 0.1 nM are achieved.

In an embodiment of the invention, the methods of this invention utilize an effective dose of the ester or amide of an alpha-ketoalkanoic acid, wherein low concentrations of the ester or amide of an alpha-ketoalkanoic acid in culture fluid or in body fluids may result. In one embodiment, the methods of this invention employ a dosage of the ester or amide of an alpha-ketoalkanoic acid such that plasma concentrations of 200 nM or less and greater than 0.1 nM are achieved.

In one embodiment, the ethyl pyruvate for administration to a subject, according to the methods of this invention, may be at a dose in the range of about 0.5 mg/kg body weight of the subject to about 20 mg/kg body weight, or alternatively lower doses of about 1 μg/kg body weight to about 1000 μg/kg body weight of the subject.

In one embodiment, the ethyl pyruvate for administration to a subject, according to the methods of this invention, may be formulated for administration in an aqueous based liquid such as phosphate buffered saline to form an emulsion, or they may be formulated in an organic liquid such as cyclodextran or dimethylsulfoxide to form a solution. The solution or emulsion may be administered by any route, known to one skilled in the art.

In one embodiment, the ester or amide of an alpha-ketoalkanoic acid for administration to a subject, according to the methods of this invention, may be formulated for administration in an aqueous based liquid such as phosphate buffered saline to form an emulsion, or they may be formulated in an organic liquid such as cyclodextran or dimethylsulfoxide to form a solution.

In one embodiment, the time of administration of a single dose of the ethyl pyruvate is within about four hours after onset of an ischemic episode. In another embodiment, the ethyl pyruvate may be administered concurrently with the onset of an ischemic episode or in another embodiment, prior to onset of ischemia. It is to be understood that the administration of ethyl pyruvate or a composition comprising the same may be at any time, which produces a therapeutic effect.

In one embodiment, the time of administration of a single dose of the ester or amide of an alpha-ketoalkanoic acid is within about four hours after onset of an ischemic episode. In another embodiment, the ester or amide of an alpha-ketoalkanoic acid may be administered concurrently with the onset of an ischemic episode or in another embodiment, prior to onset of ischemia. It is to be understood that the administration of the ester or amide of an alpha-ketoalkanoic acid or a composition comprising the same may be at any time, which produces a therapeutic effect.

The duration of exposure to the ester or amide of an alpha-ketoalkanoic acid may vary, and may in one embodiment, be a function of the particular application.

The duration of exposure to ethyl pyruvate may vary, and may in one embodiment, be a function of the particular application.

In one embodiment, the cell contacted with ethyl pyruvate is within or isolated from an organ, which is ischemic.

In one embodiment, the cell contacted with the ester or amide of an alpha-ketoalkanoic acid is within or isolated from an organ, which is ischemic.

In one embodiment, the term “ischemic” refers to tissue or organ deprivation of oxygen. In one embodiment, ischemia is as a result of underlying vascular disease. In one embodiment, cytoprotection is conferred by the methods of this invention to cells within or isolated from myocardium.

Cerebral ischemia results from decreased blood and oxygen flow. In cerebral ischemia, the individual may suffer a stroke, which may be accompanied by sudden development of a focal neurologic deficit and, in some cases, some degree of brain damage. The decreased blood flow may be due to, for example, an occlusion such as a thrombus or embolus, vessel rupture, sudden fall in blood pressure, change in the vessel lumen diameter due to atherosclerosis, trauma, aneurysm, developmental malformation, altered permeability of the vessel wall or increased viscosity or other quality of the blood.

Decreased blood flow may be due, in another embodiment, to failure of the systemic circulation and severe prolonged hypotension. Ischemic necrosis of the spinal cord may result in sensory or motor symptoms or both that can be referred to cervical, thoracic or lumbar levels of the spine.

Ischemic heart disease may result from an imbalance between myocardial oxygen supply and demand. In ischemic heart disease, the individual may suffer angina pectoris, acute myocardial infarction or sudden death The imbalance may be caused by, for example, atherosclerotic obstruction of one or more large coronary arteries, nonatheromatous coronary obstructive lesions such as embolism, coronary ostial stenosis associated with luetic aortitis, coronary artery spasm, congenital abnormalities of the coronary circulation, increased myocardial oxygen demands exceeding the normal supply capabilities as in severe myocardial hypertrophy, reduction in the oxygen carrying capacity of the blood such as in anemia, or as a consequence of inadequate cardiac profusion pressure due to hypotension from any cause.

It is to be understood that any of these conditions may be improved or treated via the methods of his invention, and represent embodiments thereof.

During glycolysis, pyruvate is continuously manufactured in the living organism, and involves a series of enzymatic reactions that occur anaerobically. The oxidative conversion of pyruvate to carbon dioxide and water releases a large amount of energy (these metabolic processes have been described in detail in biochemical texts, e.g., Lehninger 1975, pp. 417-441; Stryer 1981, pp. 254-279).

Under ischemic conditions, the small amounts of residual glucose and oxygen in the tissues are used up rapidly, within a minute or so, and then cells become energy deficient Pre-ischemic administration of glucose is detrimental because it causes lactic acidosis, resulting in tissue damage. Augmentation of ATP has been shown to result from rapid intracellular deesterification of methyl pyruvate to pyruvate and thus the generation of a significant transcellular gradient favoring filter intracellular influx of methyl pyruvate. This results in the rapid accumulation of an intracellular excess pool of pyruvate generated free of glycolytic regulators and without the obligate initial ATP investment at the glucokinase and phosphofructolinase reactions. Pyruvate is then available for pyruvate dehydrogenase conversion to acetyl CoA and citric acid cycle substrate provision. Such pyruvate stores may be particularly useful in the myocardial reperfusion phase when the citric acid cycle becomes available again, yet ATP levels are very low and require rapid repletion.

Ischemia-reperfusion injury of myocardium is a significant entity in many clinical situations, including coronary thrombolysis, percutaneous coronary interventions, cardiac surgery including coronary artery bypass graft surgery (CABG), and heart transplantation. Myocardial dysfunction and cellular injury occurs in part due to metabolic depletion during ischemia followed by ROS formation during reperfusion. The methods of the present invention provide, in one embodiment, a means of protecting myocardium against ischemia-reperfusion injury.

In one embodiment, this invention provides a method for preventing or reducing damage to the heart due to ischemia or, in another embodiment, reperfusion injury, in a subject, comprising administering ethyl pyruvate to said subject in an amount effective to suppress, inhibit or reduce the incidence of ischemic damage to the heart. In one embodiment, the ethyl pyruvate is administered to the subject prior to a surgical procedure having potential to cause cardiac ischemic damage. In another embodiment, the ethyl pyruvate is administered to the subject during a surgical procedure having potential to cause cardiac ischemic damage. In another embodiment, the ethyl pyruvate is administered to the subject following a surgical procedure having potential to result in cardiac ischemic damage. In other embodiments, said surgical procedure is coronary artery bypass graft surgery. In further embodiments, said coronary artery bypass graft surgery is off-pump coronary artery bypass graft surgery.

In another embodiment, the method for preventing or reducing damage to the heart due to ischemia or, in another embodiment, reperfusion injury, in a subject, comprises administering an ester or amide of an alpha-ketoalkanoic acid to said subject in an amount effective to suppress, inhibit or reduce the incidence of ischemic damage to the heat. In one embodiment, the ester or amide of an alpha-ketoalkanoic acid is administered to the subject prior to a surgical procedure having potential to cause cardiac ischemic damage. In another embodiment, the ester or amide of an alpha-ketoalkanoic acid is administered to the subject during a surgical procedure having potential to cause cardiac ischemic damage. In another embodiment the ester or amide of an alpha-ketoalkanoic acid is administered to the subject following a surgical procedure having potential to result in cardiac ischemic damage.

In one embodiment, the subject is in need of said treatment due to an angina condition which comprises chronic stable angina, unstable angina or post myocardial infarction angina In another embodiment, the subject is in need of such treatment due to acute myocardial infarction.

Ischemic heart disease may be readily diagnosed by one skilled in the art There may be predictive changes in the electrocardiogram, since ischemia alters electrical properties of the heart. Such changes include inversion of the T wave and displacement of the ST segment. Another important consequence of myocardial ischemia is electrical instability leading to ventricular tachycardia or ventricular fibrillation. Stress tests and coronary arteriography may also provide diagnostic information.

Since ischemic heart disease is usually a symptomatic until the extent of coronary artery blockage is well-advanced, preventative measures to control risk factors and life style patterns associated with the disease are also recommended. In patients in the symptomatic phase of the disease, meticulous attention to life patterns or risk factors must be given in an attempt to promote lesion regression or at least prevent progression. Risk factors include a positive family history of ischemic heart disease, diabetes, hyperlipidemia, hypertension, obesity and cigarette smoking. Life patterns include sedentary lifestyle, psychosocial tension and certain personality traits.

The ethyl pyruvate may be administered to a symptomatic individuals having one or more risk factors and/or life style patterns, or to individuals already in the symptomatic phase of ischemic heart disease to reduce or prevent disease progression. Additionally, ethyl pyruvate and compositions comprising the same may be administered to the following patients: those having careers that involve the safety of others (e.g., commercial airline pilots) and that present with questionable symptoms, suspicious or positive noninvasive test results, and in whom there are reasonable doubts about the state of the coronary arteries; males who are 45 or older and females who are 55 or older who will undergo valve replacement and who may or may not have clinical evidence of myocardial ischemia; and those at high risk after myocardial infarction because of the recurrence of angina, heart failure. Sequent ventricular premature contractions, or signs of ischemia in the stress test, to name just a few. Ethyl pyruvate and compositions comprising the same may be administered either separately or in combination with other cardiac drugs such as nitrates, beta-adrenergic blockers, calcium channel antagonists and/or aspirin and either separately or in combination with fibrinolytic drugs such as tissue plasminogen activator (tPA), streptokinase and urokinase. Use of ethyl pyruvate and compositions comprising the same may prolong life and/or reduce or eliminate the need for invasive procedures such as coronary arteriography and coronary artery bypass grafting.

In another embodiment, an ester or amide of an alpha-ketoalkanoic acid may be administered to a symptomatic individuals having one or more risk factors and/or life style patterns, or to individuals already in the symptomatic phase of ischemic heart disease to reduce or prevent disease progression. Additionally, the ester or amide of an alpha-ketoalkanoic acid and compositions comprising the same may be administered to the following patients: those having careers that involve the safety of others (e.g., commercial airline pilots) and that present with questionable symptoms, suspicious or positive noninvasive test results, and in whom there are reasonable doubts about the state of the coronary arteries; males who are 45 or older and females who are 55 or older who will undergo valve replacement and who may or may not have clinical evidence of myocardial ischemia; and those at high risk after myocardial infarction because of the recurrence of angina, heart failure, frequent ventricular premature contractions, or signs of ischemia in the stress test, to name just a few. The ester or amide of an alpha-ketoalkanoic acid and compositions comprising the same may be admistered either separately or in combination with other cardiac drugs such as nitrates, beta-adrenergic blockers, calcium channel antagonists and/or aspirin and either separately or in combination with fibrinolytic drugs such as tissue plasminogen activator (tPA), streptokinase and urokinase. Use of the ester or amide of an alpha-ketoalkanoic acid and compositions comprising the same may prolong life and/or reduce or eliminate the need for invasive procedures such as coronary arteriography and coronary artery bypass grafting.

Ischemia, in particular cerebral ischemia, may lead to stroke, which, in turn may cause an array of associated conditions, as described herein. In one embodiment, this invention provides a method for treating, suppressing or reducing the incidence of stroke-related injury, in a subject, comprising administering the ester or amide of an alpha-ketoalkanoic acid to said subject in an amount effective to treat, prevent or reduce stroke-related injury in said subject.

In another embodiment, this invention provides a method for treating, suppressing or reducing the incidence of stroke-related injury, in a subject, comprising administering an ester or amide of an alpha-ketoalffoic acid to said subject in an amount effective to treat, prevent or reduce stroke-related injury in said subject.

In another embodiment, this invention provides a method of treating, suppressing or inhibiting the incidence of myocardial infarction or damage caused by myocardial infarction in a subject, the method comprising administering ethyl pyruvate to a subject at risk for or undergoing myocardial infarction, in an amount effective at treating, suppressing or inhibiting the incidence of myocardial infarction or damage caused by myocardial infarction. In one embodiment, ethyl pyruvate augments cellular metabolism in myocardial cells in the subject, enhances cardiac function, limits infarct size, or a combination thereof, in said subject.

In another embodiment, this invention provides a method of treating, suppressing or minibiting the incidence of myocardial infarction or damage caused by myocardial infarction in a subject, comprising administering an ester or amide of an alpha-ketoalkanoic acid to a subject at risk for or undergoing myocardial infarction, in an amount effective at treating, suppressing or inhibiting the incidence of myocardial infarction or damage caused by myocardial infarction. In one embodiment, the ester or amide of an alpha-ketoalkanoic acid augments cellular metabolism in myocardial cells in the subject, enhances cardiac function, limits infarct size, or a combination thereof, in said subject

As exemplified hereinbelow, ethyl pyruvate treated subjects demonstrated attenuated left ventricular infarction sizes as compared to control subjects, and a statistically significant increase in cardiac function among treated subjects, as compared to controls. Moreover, the administration of ethyl pyruvate to infarcted animals brought hemodynamic parameters partially back to those levels observed among noninfrared native animals. Such activity is attributable, in one embodiment, to the combined effects of cytoprotection and increased cellular metabolism, where, in another embodiment, diminishment of myocardial infarction, and its clinical course, is accomplished with ethyl pyruvate.

Any number of diseases, disorders and conditions exist wherein the cytoprotection properties of ethyl pyruvate, or in another embodiment an ester or amide of an alpha-ketoalkanoic acid, are useful, and wherein augmentation of cellular metabolism coupled with such properties are useful. Examples of some of these diseases, disorders and conditions that benefit from the methods of this invention include: neurological and neuodegenerative diseases and conditions such as Alzheimer's disease, Parkinson's disease, amyotrophic lateral sclerosis (ALS), multiple sclerosis, peripheral neuropathy, stroke, traumatic injury, various neurological and other degenerative consequences of neurological and chest surgeries, ischemic bone disease, osteoarthritis, other types of arthritis and conditions of connective tissue and cartilage degeneration including rheumatoid, psoriatic and infectious arthtis, degenerative disorders of the eye including macular degeneration and retinal degeneration, various diseases and conditions of the heart including cardiac ischemia, myocardial infaction, chronic or acute heart failure or cardiac dysrhymias.

Efficacy of ethyl pyruvate treatment, or in another embodiment, treatment with an ester or amide of an alpha-ketoalkanoic acid may be evaluated using noninvasive clinical imaging methods such as magnetic resonance imaging (MRI) of the affected region to determine the size of the damaged area. In cerebral ischernia, it is also possible to assess neurologic deficit by performance on behavioral tests such as cognitive recognition or memory function such as the National Institutes of Heat (NIH) stroke scale.

The following are meant to provide materials, methods, and examples for illustrative purposes as a means of practicing/executing the present invention, and are not intended to be limiting.

### EXAMPLES

### Materials and Methods

Animal Care and Biosafety

Male Wistar rats weighing 250 to 350 grams were obtained from Charles River (Boston, Mass.). Food and water were provided ad libitum. Animal experiments were performed in accordance with the standard humane care guidelines of the Institutional Animal Care and Use Committee of the University of Pennsylvania, which conform to current federal guidelines.

Preparation of Treatment Solutions

Animals were divided into groups receiving Ringer's solution as a control or Ringer's solution containing 28 mmol/L ethyl pyruvate obtained from Sigma Chemicals (St “Louis, Mo.). Ringer's solution contained 130 mmol/L Na⁺, 4.0 mmol/L K⁺, 2.7 mmol/L Ca⁺², and 109 mmol/L Cl⁻ at pH 7.0.

Acute in Vivo Ischemia-Reperfusion Model

Animals (n=52) underwent induction of general anesthesia with ketamine (75 mg/kg) and xylazine (7.5 mg/kg), endotracheal intubation with a 14-gauge angiocatheter, and mechanical ventilation with 2% isofluorane maintenance anesthesia with a respirator (Hallowell EMC, Pittsfield, Mass.). Venous access for administration of treatment solutions was obtained via exposure of the right femoral vein and insertion of a 24-gauge intravenous catheter. A left thoracotomy was performed through the fourth intercostal space and the pericardium was reflected, exposing the heart. A 7-0 polypropylene suture was then placed around the left anterior descending (LAD) coronary artery and briefly snared to visually verifier the territory of myocardial ischemia. Animals were then randomized to either the control (n=26) or ethyl pyruvate group (n=26) and received a 1.5 mL/kg intravenous bolus of either Ringer's solution or ethyl pyruvate. Two minutes later, ischemia was initiated. The LAD was then occluded for 30 minutes. In a small subset of animals (control n=5, ethyl pyruvate n=5), hearts were harvested 10 minutes into the ischemia period for analysis of myocardial energetic state via ATP assay. In the other 42 animals, after the 30-minute ischemic period, just prior to reperisimon, the treatment solution was again intravenously bolused (3.0 mL/kg). Hearts were then reperfused for 30 minutes. At the end of this period, the animals were either killed and hearts were harvested for lipid peroxidation analysis (control n=6, ethyl pyruvate n=6) or animals were prepared for cardiac functional assessment and subsequent heart explanation for infarct size determination (control n=15, ethyl pyruvate n=15).

Myocardial ATP Levels

ATP levels were quantified using the commercially available EN-LITEN ATP luciferin/luciferase bioluminescence assay system (Promega, Madison, Wis.). Myocardial tissue specimens from the ischemic region were excised, immediately frozen in liquid nitrogen, and individually pulverized into a fine powder by hand grinding with a dry ice-chilled steel mortar and pestle (Manthorpe M, et al. Hum Gene Ther. 1993; 4: 419-31). Ten milligrams of myocardium were homogenized with 1 mL of pre-cooled extractant (0.1% trichloroacetic acid) and centrifuged at 4500 revolutions per minute (rpm) for 10 minutes (Stanley P. Methods Enzymol. 1986; 133: 14-22). Supernatant (100 μl) was diluted 10-fold with 50 mmol/L Tris-acetate buffer containing 2 mmol/L ethylenediaminetetraacetic acid (H 7.75). Then 100 μl of sample extract or reference standard solution was placed in a tube luminometer (Tuner Designs Luminometer TD-20/20, Promega), followed by the auto-injection of 100 μl of ATP luciferin/luciferase assay mix for ATP quantification. Luminescence was measured at a set lag time of 1 second and integration time of 10 seconds.

Myocardial Lipid Peroxidation Measurements

Myocardial lipid peroxidation was quantified using the commercially available PeroxiDetect KIT (Sigma Chemicals). Myocardial tissue specimens were obtained from the ischemic anterolateral left ventricular wall and from the nonischemic remote basal postero-septal left ventricle and immediately frozen in liquid nitrogen. Lipid soluble components were extracted from the myocardial tissue specimens using a CHCl₃/methanol extraction protocol (Yagi K, et al. Biochem. Int. 1986; 12:367-71). Briefly, 100 mg of tissue specimen was homogenized in a 2:1 volume mixture of CHCl₃ and 100% methanol at a ratio of 1 g tissue to 15 mL of the CHCl₃/methanol mixture. The homogenized material was centrifuged at 10,000 rpm for 10 minutes, after which the supernatant was clarified with 0.3 mL of 0.9% NaCl per gram of tissue and then removed. The CHCl₃ layer was evaporated with nitrogen gas, leaving lipid peroxides from the specimens contained in the methanol solvent Specimen samples (100 μL) were added to a 1-mL mixture of Fe⁺² ion and xylenol orange. Peroxides convert Fe+² to Fe⁺³, which forms a color adduct with xylenol orange spectrophotometrically detectable at 560 nm. Lipid peroxide levels were then calculated using a reference standard curve generated with defined quantities of tert-butyl hydroperoxide.

Cardiac Functional Assessment Following Ischemia and Reperfusion

Following the 30-minute reperfusion period, a median stemotomy was performed and myocardial performance was assessed in vivo (control n=15, ethyl pyruvate n=15). A fully calibrated miniature pressure/volume conductance catheter (MIKRO-TIP catheter and ARIA Pressure Volume Conductance System, Millar Esuments, Houston, Tex.) was inserted into the left ventricular cavity through the apex. Calibration consisted of the cuvette 2-point linear interpolation process and parallel conductance subtraction via the hypertonic saline method. The digitized pressure and volume signals were displayed and recorded using Chart v4.1.2 software (AD Instruments, Colorado Springs, Colo.). Multiple cardiac functional parameters were measured in the 2 groups using Cardiac Pressure Volume Analysis Software-PVAN 2.9 (Millar Instuments). Additionally, a flow probe monitor (Tran-sonic Systems, Ithaca, N.Y.) was placed around the ascending aorta to measure cardiac output. To provide a reference of baseline rat myocardial function, a separate group of 15 non infarcted native animals underwent pressure volume and cardiac output analysis.

Planimetric-Determination of Left Ventricular Infarction Size

Following the cardiac functional analysis, control and ethyl pyruvate hearts were excised and rinsed in normal saline solution (pH 7.4). The LAD was again snared and Evans blue dye was infused into the aortic root to facilitate determination of area at risk. The specimens were cut perpendicular to the long axis into 5 sections and incubated in 1% triphenyltetrazolium chloride (TTC) (Sigma Chemicals) in phosphate-buffered saline solution (pH 7.4) at 37° C. for 20 minutes. Following the incubation period, the TTC was rinsed from the sections and a 10% formalin solution was added for tissue fixation. Sections were photographed using a digital camera and were downloaded onto a desktop computer containing digital planimetry software (OpenLab, Lexington, Mass.). Infarct size as a percentage of area at risk was then measured.

Statistical Methods

Statistical analyses were performed using unpaired, 1-tailed Student/tests. All results were expressed as mean +standard error of the mean (SEM).

### Example 1

### Ethyl Pyruvate Increases ATP Production in Ischemic Myocardium

To determine ethyl pyruvate effects on myocardial high-energy phosphate levels, free radical injury, myocardial infarction, and cardiac mechanics, a model of myocardial ischemia-reperfusion injury was used. Though the proportionate degree of myocardial injury induced in the model is more pronounced than what occurs in a typical cardiac surgical procedure, the model was selected for its predictability and functionality.

In the control group, Ringer's solution was utilized to preclude any attribution of observed differences to electrolyte composition of solutions. Also, in the unlikely event that some acid-base buffering capacity of ethyl pyruvate was the only cause of observed differences, a small pilot series of control animals with lactated Ringer's solution was performed. These animals would have obtained any buffering benefit, yet myocardial function and infarct size were equivalent to Ringer's solution controls. The dose of ethyl pyruvate selected for his study was based upon a concentration of ethyl pyruvate previously optimized in an intestinal ischemia model (Sims Calif., et al., Crit Care Med. 2001; 29: 1513-8). The timing of administration of the ethyl pyruvate was designed to enhance the two purported mechanisms of action, glycolytic substrate augmentation and antioxidation.

An initial left thoracotomy was employed during the ischemic period, which enabled easy access to the LAD, ease of coronation of a large antero-lateral region of ischemic discoloration, and absence of sternal bleeding during this period. A stemotomy was then utilized for hemodynamic assessment because this allowed a more favorable angle of entry of the pressure volume conductance microcatheter into the left ventricle. In the rat, catheter insertion from a thoracotomy approach causes leftward displacement of the cardiac apex with resultant torsion on the heart and hemodynamic compromise similar to that seen clinically when displacing the heart during off-pump grafting of lateral coronary arteries. Furthermore, a stemotomy facilitated easier access to the inferior vena cava for load variation to assess pressure-volume relationships.

To determine whether ethyl pyruvate is involved in glycolytic substrate augmentation, tissue AT? levels were assayed in the ischemic myocardial territory 10 minutes after LAD snaring. This period of time was chosen to permit depletion of potentially confounding myocardial high-energy phosphate reserves as well as to ensure dependence upon anaerobic glycolysis. Excess exogenous pyruvate could liberate NAD+ for more proximal glycolytic pathway generation of ATP.

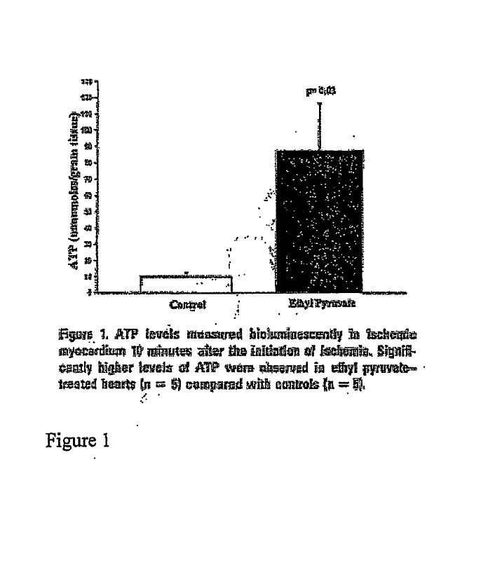

Analysis of AT? levels in ischemic hearts revealed a marked increase in animals receiving ethyl pyruvate compared with control animals (FIG. 1). The ischemic region of control hearts contained 10.0±2.4 nmol/g, whereas the ischemic region of ethyl pyruvate hearts contained 87.6±29.2 nmol/g (P=0.03).

### Example 2

### Ethyl Pyruvate Treatment Diminishes Myocardial Lipid Peroxidation

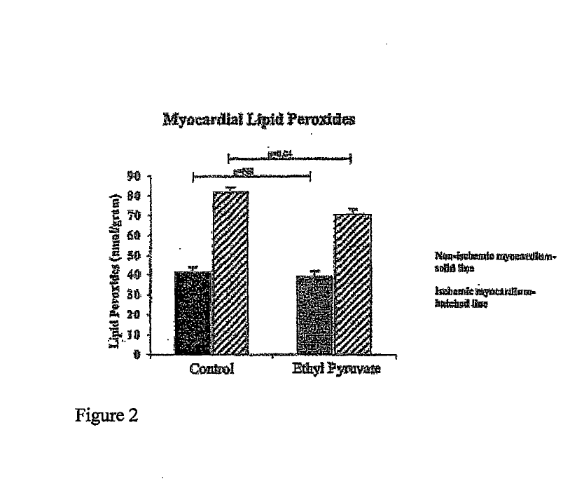

Two experiments were conducted to determine myocardial lipid peroxidation, which is a measure of oxidative stress. Spectrophotometric quantification of lipid peroxides in myocardium exposed to ischemia and reperfiusion, and in nonischemic myocardium, which served as an internal, control, is represented in FIGS. 2 (n=6 mice per group) and 3 (n=5 mice per group). Lipid peroxide levels in the nonischemic myocardium did not differ significantly between animals treated with ethyl pyruvate (42.3±4.8 nmol/g; n=6) and the control, Ringer's solution (37.5±5.1 nmol/g; n=6), implying equivalent assay conditions between control and ethyl pyruvate hearts. Ischemia significantly increased the level of lipid peroxidation from 37.5±5.1 nmol/g to 89.5±3.0 nmol/g in control hearts (P<0.001). When comparing ischemic myocardium in ethyl pyruvate hearts with control hearts, there was a statistically significant decrement in lipid peroxidation with ethyl pyruvate administration (70.4±2.6nmol/g vs. 81.8±2.4nmol/g, p=0.04, and 63.8±3.3 vs 89.5±3.0 nmol/g P<0.001).

### Example 3

### Ethyl Pyruvate Treatment Decreases Myocardial Infarction

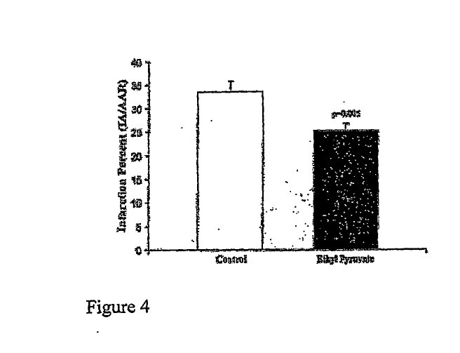

Macroscopic analysis of TTC-stained cross sections following 30 minutes of ischemia and 30 minutes of reperfiusion demonstrated attenuated left ventricular infarction sizes in animals treated with ethyl pyruvate (n=15) as compared with the control group (n=is; FIG. 4). Ethyl pyruvate animals had 25.3%±1.5% of the left ventricular area at risk infarcted as compared with 33.6%±2.1% in control animals (P=0.005).

### Example 4

### Ethyl Pyruvate Treatment Improves Cardiac Function

In order to determine overall effects of ethyl pyruvate on cardiac function, a number of parameters were evaluated over the course of induced ischemia, and reperfusion, as a function of time.

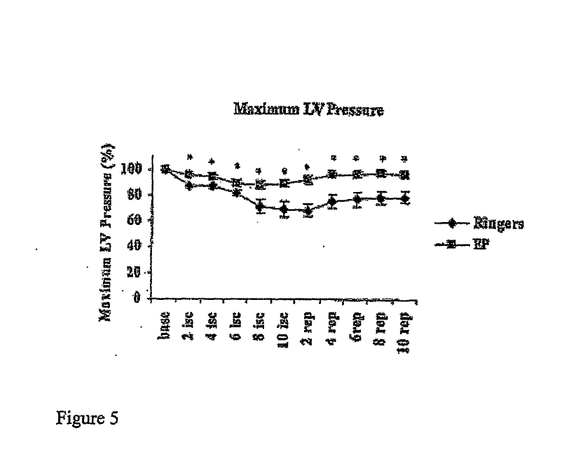

One parameter measured was maximum left ventricular (LV) pressure, as compared to baseline. Maximum LV pressure was significantly greater throughout ischemia and reperftrsion in the ethyl pyruvate treated group, as compared to controls (FIG. 5).

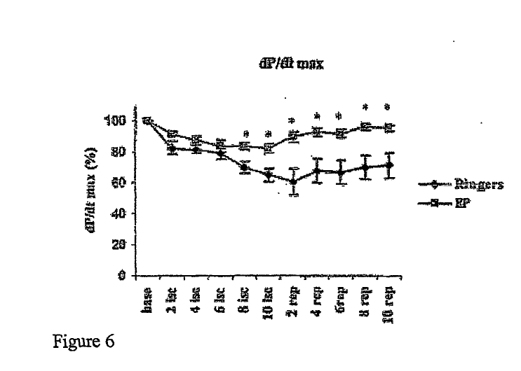

Another parameter measured was contractility. As seen in FIG. 6, the dP/dt maximum was significantly greater after 8 minutes of ischemia and throughout reperision in the ethyl pyruvate treatment group compared to controls. Cardiac output expressed as a percentage of baseline.

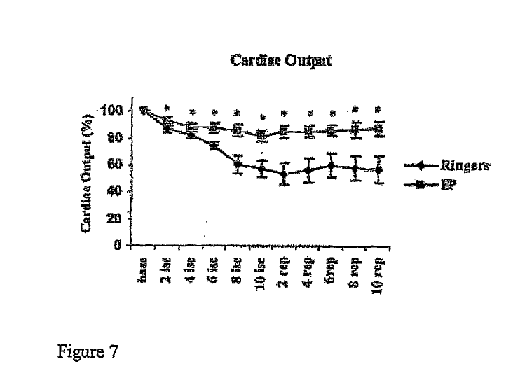

In addition, cardiac output was found to be significantly greater throughout ischemia and reperfulsion in the ethyl pyruvate treatment group, as compared to controls (FIG. 7).

Table 1 displays data from a second group of animals, indicating results obtained for heart rate and multiple pressure and volume parameters for native animals (n=15) as a reference, Ringer's control animals (n=15), and ethyl pyruvate animals (n=15).

TABLE 1
Hemodynamic Data

| | | | | P Value |
|---|---|---|---|---|
| | | | Ethyl | (Control |
| | Native | Control | Pyruvate | Vs. Ethyl |
| Parameter | (n = 15) | (n = 15) | (n = 15) | Pyruvate) |
| Heart rate | 210 ± 7.6 | 176 + 14.4 | 183 ± 9.5 | NS |
| (bpm) | | | | |
| Maximum | 98.7 ± 2.9 | 73.5 ± 2.5 | 86.6 ± 2.9 | <001 |
| pressure | | | | |
| (mm Hg) | | | | |
| Maximum | 4974 + 199 | 2703 ± 175 | 3518 + 243 | .005 |
| dP/dt | | | | |
| (mm Hg/s) | | | | |
| Minimum | −3940 ± 258 | 1745 ± 170 | 2841 + 329 | .003 |
| dP/dt | | | | |
| (mm Hg/s) | | | | |
| Maximum | 3919 ± 321 | 2120 ± 287 | 3097 ± 479 | .04 |
| dV/dt | | | | |
| (j/i/s) | | | | |
| Ejection | 47.1 + 3.3 | 31.4 ± 4.1 | 41.9 ± 3.8 | .03 |
| fraction | | | | |
| (%) | | | | |
| Cardiac | 33.0 ± 1.8 | 22.7 ± 1.3 | 26.7 + 0.9 | .01 |
| output | | | | |
| (mL/min) | | | | |

bpm, Beats per minute; dP/dt, rate of pressure rise; dV/dtᵣ

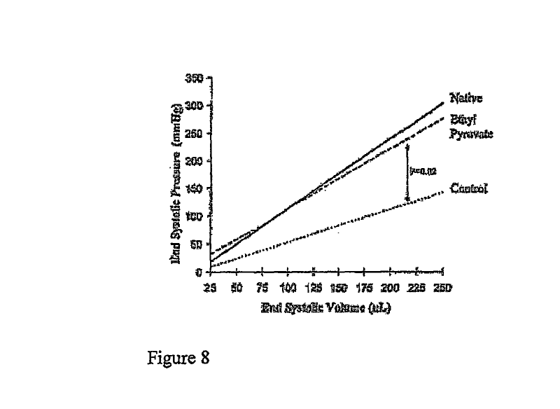

Statistical comparisons between control and ethyl pyruvate animals are displayed. Heart rates were equivalent. In all pressure and volume parameters 20 measured, there was a statistically significant increase in cardiac fraction among ethyl pyruvate animals compared with controls. The administration of ethyl pyruvate to infarcted animals brought hemodynamic parameters partially back to those levels observed among non infarcted native animals. Bind systolic pressure volume relationships were assessed and represented as a regression composite for each of the 3 groups (FIG. 8). Ethyl pyruvate animals exhibited significantly improved myocardial function compared with control animals, approaching native baseline values.

What is claimed is:

1. A method for conferring cytoprotection to a cell, comprising contacting said cell with an amount of ethyl pyruvate, effective to confer cytoprotection.

2. The method of claim 1, wherein said cell is within or isolated from an organ which is ischemic.

3. The method of claim 1, wherein said cell is within or isolated from an organ, which is at risk of ischemia.

4. The method of claim 1, wherein contacting said cell is conducted in vivo.

5. The method of claim 1, wherein said cell is of cardiac, intestinal or neural origin.

6. The method of claim 5, wherein said cell is within or isolated from myocardium.

7. The method of claim 1, wherein contacting said cell is conducted ex-vivo.

8. The method of claim 1, wherein said cell is implanted within a subject following contact with said effective amount of ethyl pyruvate.

9. The method of claim 1, wherein said effective amount of ethyl pyruvate inhibits or prevents oxidative damage to said cell.

10. The method of claim 1, wherein said effective amount of ethyl pyruvate augments cellular metabolism.

11. The method of claim 1, wherein said cell is within a population of cells which will be transplanted to a subject.

12. A method for conferring cytoprotection to a cell, comprising contacting said cell with an amount of an ester of an alpha-ketoalkanoic acid effective to confer cytoprotection.

13. The method of claim 12, wherein said ester is a C₃-C₈ straight-chained or branched alpha-ketoalkanoic acid.

14. The method of claim 12, wherein said ester is an alkyl, aralkyl, alkoxyalkyl or carbalkoxyalkyl ester.

15. The method of claim 14, wherein said ester is an ethyl ester.

16. The method of claim 12, wherein said cell is within or isolated from an organ which is ischemic.

17. The method of claim 12, wherein said cell is within or isolated from an organ, which is at risk of ischemia.

18. The method of claim 12, wherein contacting said cell is conducted in vivo.

19. The method of claim 18, wherein said cell is of cardiac, intestinal or neural origin.

20. The method of claim 12, wherein said cell is within or isolated from myocardium.

21. The method of claim 12, wherein contacting said cell is conducted ex-vivo.

22. The method of claim 12, wherein said cell is implanted within a subject following contact with said effective amount of said ester of an alpha-ketoalkanoic acid.

23. The method of claim 12, wherein said effective amount of said ester of an alpha-ketoalkanoic acid augments cellular metabolism.

24. The method of claim 12, wherein said cell is when a population of cells which will be transplanted to a subject.

25. A method for treating or reducing damage to the heart due to ischemia or reperfusion injury in a subject, comprising administering ethyl pyruvate to said subject in an amount effective to treat or reduce ischemic damage to the heart

26. A method for treating or reducing damage to the heart due to ischemia or reperfusion injury in a subject, comprising administering an ester of an alpha-ketoalkanoic acid to said subject in an amount effective to treat or reduce ischemic damage to the heart.

27. The method of claim 26 wherein said ester is a C₃-C₈ straight-chained or branched alpha-ketoalkanoic acid.

28. The method of claim 26, wherein said ester is an alkyl, aralkyl, alkoxyalkyl or carbalkoxyalkyl ester.

29. The method of claim 26, wherein said ester is an ethyl ester.

30. The method of claim 29, wherein said ester is ethyl pyruvate.

31. The method of claim 26 wherein said cells are myocardial cells.

32. The method of claim 26, wherein said ester of an alpha-ketoalkanoic acid is administered to said subject prior to a surgical procedure having potential to cause cardiac ischemic damage.

33. The method of claim 26, wherein said ester of an alpha-ketoalkanoic acid is administered to said subject during a surgical procedure having potential to cause cardiac ischemic damage.

34. The method of claim 26, wherein said ester of an alpha-ketoalkanoic acid is administered to said subject following a surgical procedure having potential to result in cardiac ischemic damage.

35. The method of claim 26, wherein said subject is in need of said treatment due to an angina condition which comprises chronic stable angina, unstable angina or post myocardial infarction angina.

36. The method of claim 26, wherein said subject is in need of such treatment due to acute myocardial infarction.

37. The method of claim 33, wherein said surgical procedure is coronary artery bypass surgery,

38. The method of claim 33, wherein said surgical procedure is cardiopulmonary bypass surgery.

39. The method of claim 33, wherein said surgical procedure is off-pump coronary artery bypass surgery.

40. The method of claim 33, wherein said procedure is conducted in the thoracic cavity.

41. A method for treating or reducing stroke-related injury, in a subject comprising administering ethyl pyruvate to said subject in an amount effective to treat or reduce stroke-related injury in said subject.

42. A method for treating or reducing stroke-related injury, in a subject, comprising administering an ester of an alpha-ketoalkanoic acid to said subject in an amount effective to treat or reduce stroke-related injury in said subject.

43. The method of claim 53, wherein said ester of an alpha-ketoalkanoic acid augments cellular metabolism in a neuron in said subject.

44. A method of treating, or inhibiting myocardial infarction or damage caused by myocardial infarction in a subject, the method comprising administering an amount of ethyl pyruvate to a subject at risk for or undergoing myocardial infarction, effective to treat or inhibiting myocardial infarction or damage caused by myocardial infarction.

45. The method of claim 44, wherein said ester of an alpha-ketoaLkiaoic acid augments cellular metabolism in myocardial cells in said subject.

46. The method of claim 44, wherein said ester of an alpha-ketoalkanoic acid enhances cardiac function, limits infarct size, or a combination thereof, in said subject.

47. Use of a composition comprising ethyl pyruvate ester of an alpha-ketoalkanoic acid for treating or preventing ischemic damage in an organ in a subject.

48. Use of a composition comprising an ester of an alpha-ketoalkanoic acid for treating or preventing ischemic damage in an organ in a subject.

49. The use of claim 48, wherein said ischemic damage is to the heart, intestine, or central nervous system.

50. Use of a composition comprising an ester of an alpha-ketoalkanoic acid as a cytoprotection agent for cells or organs for transplantation.
